(19) United States
(12) Patent Application Publication
(10) Pub. No.: US 2011/0041857 A1
(43) Pub. Date: Feb. 24, 2011

(54) FIBROBLAST DERIVED STEM CELLS

(75) Inventors: Adam J.T. Schuldt, Stony Brook, NY (US); Peter R. Brink, Setauket, NY (US); Ira S. Cohen, Stony Brook, NY (US); Michael R. Rosen, New York, NY (US); Richard B. Robinson, Cresskill, NJ (US); Glenn Gaudette, Holden, MA (US)

Correspondence Address: DITTHAVONG MORI & STEINER, P.C., 918 Prince Street, Alexandria, VA, 22314, US

(73) Assignees: The Trustees of Columbia University In The City of New York, New York, NY (US); The Research Foundation of State University of New York, Albany, NY (US); Worchester Polytechnic Institute, Worcester, MA (US)

(21) Appl. No.: 12/744,568

(22) PCT Filed: Nov. 26, 2008
(86) PCT No.: PCT/US08/84876

§ 371 Date: Nov. 15, 2010

Related U.S. Application Data
(60) Provisional application No. 61/004,335, filed on Nov. 26, 2007.

(51) Int. Cl. A61B 19/00; C12N 5/02; C12N 5/074; C12N 5/10; A61K 35/12; A61P 9/10

(52) U.S. Cl. 128/898; 435/377; 435/325; 424/93.7

(57) ABSTRACT

The present invention provides methods and compositions relating to the production of stem cells, derived from dedifferentiated fibroblasts, and the use of such stem cells for treatment of a variety of different disorders and conditions. The invention is based on the surprising discovery that a population of stem cells, capable of differentiating into a variety of different cell types, can be generated by culturing fibroblasts under selective culture conditions.

### FEDERAL FUNDING

This invention was made with government support under grant/contract number HL28958 awarded by NHLBI. The government has certain rights to the invention.

### INTRODUCTION

The present invention provides methods and compositions relating to the production of stem cells, derived from dedifferentiated fibroblasts, and the use of such stem cells for treatment of a variety of different disorders and conditions. The invention is based on the surprising discovery that a population of stem cells, capable of differentiating into a variety of different cell types, can be generated by culturing fibroblasts under selective culture conditions.

### BACKGROUND OF INVENTION

The use of stem cells represents a promising approach for therapeutic intervention for a wide variety of different diseases or conditions given the ability of such stem cells to differentiate into various and diverse specific types of cells such as bone marrow, neuronal, cardiovascular, hepatic, kidney, skin, etc.

To date, much of the work regarding stem cells has focused on the use of embryonic stem cells to provide treatments for a variety of diseases. However, the use of embryonic stem cells creates moral and ethical dilemmas because the basic technology requires a supply of stem cells from embryos which often results in the death of the embryo. The present invention provides a simple and non-controversial method for deriving stem cells which are capable of functioning as multipotent or pluripotent stem cells.

### SUMMARY OF THE INVENTION

The present invention provides methods and compositions relating to the use of stem cells derived from dedifferentiated fibroblasts for treatment of a variety of different disorders. The invention is based on the discovery that a cardiogenic, or adipocytic, population of cells can be produced by culturing fibroblasts under selective conditions.

In one embodiment of the present invention, a method is provided for the production of stem cells, of fibroblast origin, comprising: (a) generating a culture of fibroblasts from a tissue sample; and (b) culturing the fibroblasts in a suitable culture vessel containing cell culture medium for a time sufficient to allow the dedifferentiation of said fibroblasts, wherein said cell medium promotes the growth of the fibroblast in suspension.

In another embodiment of the invention, a composition is provided comprising a stem cell, derived from a dedifferentiated fibroblast, and produced according to the method of the present invention. In another embodiment of the invention, a composition is provided comprising a stem cell derived from a dedifferentiated fibroblast, and produced according to the present invention, wherein the stem cell has been induced into a more terminally differentiated cell prior to implantation into a host in need of said terminally differentiated cell. Said compositions may further comprise a pharmaceutically acceptable carrier.

In yet another embodiment, the stem cells of the present invention may be used directly in therapeutic methods for treating a variety of disease states and conditions, or alternatively, the stem cells may be further differentiated into cells of a desired lineage and those cells may be used in therapeutic methods. The present invention includes and provides methods for treating diseases such as, for example, liver cirrhosis, pancreatic insufficiency, treating acute or chronic kidney failure, heart disease, pulmonary disorders, stroke and skin disorders.

### BRIEF DESCRIPTION OF THE FIGURES

FIG. 1. Representative images showing the formation of spheres from fibroblasts over time. FIG. 1A shows that the cells begin as an evenly distributed monolayer. FIG. 1B demonstrates that by 24 hours after plating, the cells have undergone a morphological change. FIG. 1C shows that at 48 hours, the cells have begun to migrate toward one another, forming clusters. FIG. 1D shows that by 72 hours, the clusters pull together into spheres. Relatively few fibroblasts remain in the monolayer, indicating that most have entered spheres. FIG. 1E demonstrates that after 4 days, the spheres are well-defined and growing in size.

FIG. 2. Cardiac differentiation of canine cardiosphere-derived cells (CFDCs). Cardiac explants were cultured for several days, during which time fibroblasts egressed from the explants and formed a monolayer on the bottom of the culture plate. The fibroblasts were collected and replated on a poly-D-lysine coated plate. After 2-3 days, the cells pulled together into clusters and lifted off of the plate as floating spheres (cardiac fibrospheres). These spheres were collected, dissociated to single cardiac fibrosphere-derived cells (CFDCs), and labeled with DiI (orange). The labeled CFDCs were then placed in co-culture with neonatal rat ventricular myocytes (NRVMs) in segregated areas of a coverglass and allowed to share media for five days. The cells were then fixed and stained for the cardiac marker sarcomeric α-actinin (green). Nuclei were stained with DAPI (blue). FIG. 2A. DiI+cells within the CFDC area of the coverglass. FIG. 2B. Sarcomeric α-actinin staining of the same cell showing distinct sarcomeric structure. FIG. 2C. Merged image showing co-localization of the DiI and sarcomeric α-actinin staining in a mono-nucleated CFDC. The presence of one nucleus indicates that cell fusion is not responsible for co-localization of the DiI and sarcomeric α-actinin.

FIG. 3. Adipogenic differentiation of canine dermal fibrosphere-derived cells (DFDCs). Canine dermal explants were kept in culture for several days until a monolayer of fibroblasts covered the bottom of the culture plate. The fibroblasts were collected and replated in poly-D-lysine coated plates, where they formed floating clusters of cells (fibrospheres) after 2-3 days. The fibrospheres were collected, dissociated and plated at confluence for adipogenic differentiation using a commercially available kit (Lonza, Walkersville, Md.). Adipogenic differentiation of DFDCs (FIG. 3D) and human mesenchymal stem cells (MSCs; FIG. 3B) is indicated by the appearance of fat vacuoles which stain red with Oil Red-0 stain. Human MSC and canine DFDC control cells (FIGS. 3A and C, respectively) that were not exposed to adipogenic media are also shown after Oil Red-0 staining.

### DETAILED DESCRIPTION OF THE INVENTION

The present invention relates to adult stem cells derived from dedifferentiated fibroblasts, as well as methods for their production and use for treatment of a variety of different diseases and conditions. Such dedifferentiated cells, for purposes of the present invention, are referred to hereafter as “stem cells”. The term “dedifferentiation” is understood to the person skilled in the art, to specify the regression of an already specialized (differentiated) cell to a stem cell which has the potential to differentiate into a number of different cell types. Surprisingly, as described herein, it has been demonstrated that the methods provided by the present invention, lead to the dedifferentiation of fibroblasts into stem cells. The stem cells produced in this way can be transformed into a number of different cells indicating the multipotency of the derived cells.

The method according to the present invention provides a completely safe and efficient way to generate stem cells. In addition, in the case of autologous use, the stem cells provided for the treatment of a disease or condition, do not give rise to any immunological problems such as cell rejection, as cells and recipient are preferably genetically identical.

The present invention provides a method for the production of stem cells of human fibroblast origin comprising: (a) generating a culture of fibroblasts from a tissue sample; (b) culturing of the fibroblasts in a suitable culture vessel containing cell culture medium for a time sufficient to allow the dedifferentiation of said cells, wherein said cell medium promotes cell growth in suspension; and (c) obtaining the dedifferentiated fibroblasts, by separating the cells from the culture medium.

A starting material for the process according to the invention is tissue containing fibroblasts. These are preferably autologous fibroblasts, i.e., fibroblasts, which originate from the tissue of the patient to be treated with the stem cells according to the invention or the target cells produced from these.

Methods well know to those of skill in the art may be used to obtain a culture of fibroblast. For example, tissue samples may be collected, minced, and treated with trypsin. The cells are then transferred to cell culture plates and kept at 37° C. The culture plates may be treated with fibronectin or other coatings to improve attachment of the tissue explants to the plates. The explants are maintained in culture with regular media changes. After several days, fibroblasts egress from the explants and form a monolayer on the bottom of the plate. These fibroblasts may be collected by lightly treating the plate with trypsin and gently washing media over the bottom of the plate. The explants can be left in the plate to produce more cells for future harvests.

The fibroblasts collected from the explants are then resuspended in any media known to be capable of supporting the growth of fibroblasts. In a specific embodiment of the invention, Fibrosphere Growth Medium (35% DMEM, 65% IMDM/F12 mix with 3.5% fetal bovine serum, 1×B-27 (Invitrogen), 1% penicillin-streptomycin, 1% L-glutamine) can be used. Additionally, the media may be supplemented with one or more of the following growth factors: thrombin, basic fibroblast growth factor, epithelial growth factor, and cardiotrophin-1. The resuspended cells are transferred to culture vessel which promote cell growth in suspension. In a non-limiting embodiment of the invention, the culture vessel may be coated with poly-D-lysine. The cells are then maintained in culture for 4 days or more. During this incubation period, the culture conditions of the invention promote the aggregation of cells into a three dimensional geometric conformation, such as spherical structures. The spheres are collected and treated with trypsin or other enzymatic treatments to produce a single-cell suspension.

The dissociated cells can then be treated with standard cell differentiation kits to induce differentiation along the desired lineages. Alternatively, the disassociated cells may be co-cultured with cells or tissue to induce differentiation along a desired lineage. For example, the cells may be co-culured with cells or tissue derived from liver, pancreas, heart, kidney, lung, or nervous system. In a specific embodiment of the invention, the dissociated cells are or co-cultured with neonatal cardiomyocytes or cardiac explants from laboratory animals to drive them along a cardiac lineage.

The process according to the invention surprisingly leads to the dedifferentiation of fibroblasts, resulting in a population of stem cells capable of differentiating into a variety of different cell types. The stem cells obtained in this way, floating freely in the medium, can either be directly transferred to a culture media capable of “reprogramming” the stem cells to differentiate into a desired cell type, or alternatively, may be cultured in the presence of a cytokine or LIF (leukemia inhibitory factor) in order to avoid premature loss of mutipotency or totipotency. Alternatively, the cells can be deep-frozen for storage purposes.

The stem cells produced using the methods of the present invention can be reprogrammed into any desired cell type. Processes for reprogramming stem cells are known in the state of the art, cf. for example Weissman I. L., Science 287: 1442-1446 (2000) and Insight Review Articles Nature 414: 92-131 (2001), and the handbook “Methods of Tissue Engineering”, Eds. Atala, A., Lanza, R. P., Academic Press, ISBN 0-12-436636-8; Library of Congress Catalog Card No. 200188747.

In one embodiment of the invention the stem cells are used for the in-vitro production of cells (target cells) and tissue (target tissue) of a particular type. Accordingly the invention provides methods of producing target cells and/or target tissue from dedifferentiated cells of fibroblastic origin.

The stem cells according to the invention can be differentiated in vitro into desired target cells, such as for example, cardiomyocytes, adipocytes, neurons and glia cells in a medium which contains factors, i.e, “differentiation agents”, known to promote the differentiation of stem cells into cells of the desired lineage.” The term “differentiation agent” is used to describe agents which may be added to cell culture containing stem cells which will induce the cells to a desired cellular phenotype. Differentiation agents include, for example, growth factors such as fibroblast growth factor (FGF), transforming growth factors (TGF), ciliary neurotrophic factor (CNTF), bone-morphogenetic proteins (BMP), leukemia inhibitory factor (LIF), glial growth factor (GGF), tumor necrosis factors (TNF), interferon, insulin-like growth factors (IGF), colony stimulating factors (CSF), KIT receptor stem cell factor (KIT-SCF), interferon, triiodothyronine, thyroxine, erythropoietin, thrombopoietin, silencers, (including glial-cell missing, neuron restrictive silencer factor), SHC(SRC-homology-2-domain-containing transforming protein), neuroproteins, proteoglycans, glycoproteins, neural adhesion molecules, Wnt5a, cardiotropin and other cell-signalling molecules and mixtures, thereof. Additionally, commercially available differentiation kits capable of inducing stem cell differentiation along the desired lineage may be used.

Alternatively, the stem cells may be grown in the supernatant of the culture medium, in which the target cell type or tissue has been incubated. This supernatant is referred to hereafter as “conditioned medium”. In an alternative embodiment of the invention, the stem cells of the invention are used directly for the in-vivo production of target cells and target tissue.

Prior to administration of the stem cells of the invention, the cells may be genetically engineered using techniques well known in the art to express proteins that enhance the ability of such cells to differentiate and/or proliferate or proteins that provide a therapeutic benefit. Such techniques include, for example, in vitro recombinant DNA techniques, synthetic techniques, and in vivo genetic recombination. (See, for example, the techniques described in Sambrook J et al. 2000. Molecular Cloning: A Laboratory Manual (Third Edition), and Ausubel et al (1996) Current Protocols in Molecular Biology John Wiley and Sons Inc., USA). Any of the methods available in the art for gene delivery into a host cell can be used according to the present invention to deliver genes into the stem cells of the invention. Such methods include electroporation, lipofection, calcium phosphate mediated transfection, or viral infection. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215.

The present invention further provides pharmaceutical compositions comprising the stem cells of the invention and a pharmaceutically acceptable carrier. Pharmaceutically acceptable carriers are well known to those skilled in the art and include, but are not limited to, 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

The stem cells of the invention can also be incorporated or embedded within scaffolds that are recipient-compatible and which degrade into products that are not harmful to the recipient. These scaffolds provide support and protection for stem cells that are to be transplanted into the recipient subjects. Natural and/or synthetic biodegradable scaffolds are examples of such scaffolds.

A variety of different scaffolds may be used successfully in the practice of the invention. Such scaffolds are typically administered to the subject in need of treatment as a transplanted patch. Preferred scaffolds include, but are not limited to biological, degradable scaffolds. Natural biodegradable scaffolds include collagen, fibronectin, and laminin scaffolds. Suitable synthetic material for a cell transplantation scaffold must be biocompatible to preclude migration and immunological complications, and should be able to support extensive cell growth and differentiated cell function. It may also be resorbable, allowing for a completely natural tissue replacement. The scaffold should be configurable into a variety of shapes and should have sufficient strength to prevent it from collapsing or from pressure-induced bursting upon implantation. Recent studies indicate that the biodegradable polyester polymers made of polyglycolic acid fulfill all of these criteria, as described by Vacanti, et al. J. Ped. Surg. 23:3-9 (1988); Cima, et al. Biotechnol. Bioeng. 38:145 (1991); Vacanti, et al. Plast. Reconstr. Surg. 88:753-9 (1991). Other synthetic biodegradable support scaffolds include synthetic polymers such as polyanhydrides, polyorthoesters, and polylactic acid.

Attachment of the cells to the scaffold polymer may be enhanced by coating the polymers with compounds such as basement membrane components, agar, agarose, gelatin, gum arabic, collagens types I, II, III, IV and V, fibronectin, laminin, glycosaminoglycans, mixtures thereof, and other materials known to those skilled in the art of cell culture. Additionally, such scaffolds may be supplemented with additional components capable of stimulating cell proliferation or differentiation. Additionally, angiogenic and other bioactive compounds can be incorporated directly into the support scaffold so that they are slowly released as the support scaffold degrades in vivo. Factors, including nutrients, growth factors, inducers of proliferation or differentiation, products of secretion, immunomodulators, inhibitors of inflammation, regression factors, biologically active compounds which enhance or allow ingrowth of nerve fibers, hyaluronic acid, and drugs, which are known to those skilled in the art and commercially available with instructions as to what constitutes an effective amount, from suppliers such as Collaborative Research and Sigma Chemical Co. Similarly, polymers containing peptides such as the attachment peptide RGD (Arg-Gly-Asp) can be synthesized for use in forming scaffolds (see e.g U.S. Pat. Nos. 4,988,621, 4,792,525, 5,965,997, 4,879,237 and 4,789,734).

In another example, the stem cells of the invention may be transplanted in a gel scaffold (such as Gelfoam from Upjohn Company), which polymerizes to form a substrate in which the cells can grow. A variety of encapsulation technologies have been developed (e.g. Lacy et al., Science 254:1782-84 (1991); Sullivan et al., Science 252:718-712 (1991); WO 91/10470; WO 91/10425; U.S. Pat. No. 5,837,234; U.S. Pat. No. 5,011,472; U.S. Pat. No. 4,892,538). During open surgical procedures, involving direct physical access to the damaged tissue and/or organ, all of the described forms of stem cell delivery preparations are available options. These cells can be repeatedly transplanted at intervals until a desired therapeutic effect is achieved.

The stem cells of the present invention are useful in a wide range of therapeutic applications for cellular regenerative/reparative therapy. For example, the stem cells of the present invention can be used to replenish stem cells in mammals whose natural stem cells have been depleted due to, for example age, chemotherapy or radiation therapy. In another non-limiting example, the stem cells of the present invention can be used in organ regeneration and tissue repair. In other embodiments of the present invention, the stem cells can be used to treat dystrophic muscles and muscles damaged by ischemic events such as myocardial infarcts or to ameliorate scarring following a traumatic injury or surgery.

The methods of the invention, comprise administration of the stem cells of the invention in a pharmaceutically acceptable carrier, for treatment of a variety of different disorders. “Administering” shall mean delivering in a manner which is effected or performed using any of the various methods and delivery systems known to those skilled in the art. Administering can be performed, for example, pericardially, intracardially, subepicardially, transendocardially, via implant, via catheter, intracoronarily, intravenously, intramuscularly, subcutaneously, parenterally, topically, orally, transmucosally, transdermally, intradermally, intraperitoneally, intrathecally, intralymphatically, intralesionally, epidurally, or by in vivo electroporation. Administering can also be performed, for example, once, a plurality of times, and/or over one or more extended periods.

In certain embodiments of the invention, focal delivery may be required. Several methods to achieve focal delivery are feasible; for example, the use of catheters and needles, and/or growth on a matrix and a “glue.” Whatever approach is selected, the delivered cells should not disperse from the target site.

In another embodiment of the invention, delivery of cells may be made using a method which allows for the dispersion and homing of the stem cells to a target site in the treated subject. For example, in instances were the stem cells are to be used to repopulate bone marrow, it would be desirable to administer said cells in such a manner to permit mobilization and production of circulating blood cells.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carvers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E.W. Martin. Such compositions will contain a therapeutically effective amount of the therapeutic compound, preferably in purified form, together with a suitable amount of carrier so as to provide the form for proper administration to the patient. The formulation should suit the mode of administration.

The appropriate concentration of the composition of the invention which will be effective in the treatment of a particular disorder or condition will depend on the nature of the disorder or condition, and can be determined by one of skill in the art using standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

In instances were cardiac disorders are treated, the progress of the recipient receiving the treatment may be determined using assays that are designed to test cardiac function. Such assays include, but are not limited to ejection fraction and diastolic volume (e.g., echocardiography), PET scan, CT scan, angiography, 6-minute walk test, exercise tolerance and NYHA classification.

In addition to the therapeutic uses described herein, the stem cells of the invention may be utilized to study the biological basis and progression of diseases and conditions, including for example, genetic disorders. For such uses, the stem cells are derived from fibroblasts isolated from a subject suffering from such disorders. In yet another embodiment of the invention, the cells may be used as a model system for studying the basis of cell dedifferentiation and differentiation.

### TREATMENT OF CARDIAC DISORDERS

The present invention provides methods and compositions relating to the use mesenchymal stem cells for treatment of cardiac disorders, are disclosed in PCTUS07/16429, which is incorporated by reference in its entirety. Such disclosed methods may be used equally as well, using the stem cells of the present invention. For example, compositions comprising the stem cells of the present invention may be used as a source for cardiomyocytes and/or angiogenesis (i.e., endothelial and smooth muscle cells). Alternatively, the cells may be used for regenerating myocardium through stimulation of native cardiomyocyte proliferation. Specifically, the invention relates to the use of stem cells to promote an increase in the number of cells in the myocardium through increased proliferation of native cardiac progenitor cells resident in the myocardium; stimulation of myocyte proliferation; and stimulation of differentiation of host cardiac progenitor stem cells into cardiac cells, for example. Such an increase in cell number results predominantly from stimulation of the native myocardium cells by factors produced by the administered of stem cells. In another embodiment of the invention, scaffolds designed for implantation, as described above, may be engineered to contain exogenously added stem cells, which are capable of stimulating cardiomyocyte proliferation.

In an embodiment of the invention, the stem cells of the invention may comprise an exogenous molecule including, but are not limited to, oligonucleotides, polypeptides, or small molecules, and wherein said stem cell is capable of delivering said exogenous molecule to an adjacent cell. Delivery of the exogenous molecule to adjacent cells may be used to stimulate cardiomyocyte proliferation, cardiac repair or provide biological pacemaker activity.

The present invention provides a method of delivering an oligonucleotide, protein or small molecule into a target cell comprising: (i) introducing the oligonucleotide, protein, or small molecule into a stem cell and (ii) contacting the target cell with the stem cell under conditions permitting the stem cell to form a gap junction channel with the target cell, whereby the oligonucleotide, protein, or small molecule is delivered into the target cell from the stem cell.

In yet another embodiment of the invention, the stem cells of the invention may be genetically engineered to express a protein or oligonucleotide of interest capable of stimulating cardiomyocyte proliferation, cardiac repair or providing biological pacemaker activity.

In a specific embodiment of the invention, the stem cells of the invention are engineered to functionally expresses a hyperpolarization-activated, cyclic nucleotide-gated (HCN) ion channel, and wherein expression of the HCN channel is effective to induce a pacemaker current in said cell. In an embodiment of the invention, the expressed HCN channel is a mutant or chimeric HCN channel. Chimeric HCN channels are those HCN channels comprising an amino terminal portion, an intramembrane portion, and a carboxy terminal portion, wherein the portions are derived from more than one HCN isoform. In a preferred embodiment of the invention, the chimeric or mutant HCN channel provides an improved characteristic, as compared to a wild-type HCN channel, selected from the group consisting of faster kinetics, more positive activation, increased levels of expression, increased stability, enhanced cyclic nucleotide responsiveness, and enhanced neurohumoral response. Such stem cells may also be engineered to functionally expresses a MiRP1 beta subunit along with an HCN channel.

In addition, this invention provides a biological pacemaker comprising the stem cells of the invention which functionally expresses an HCN ion channel or a mutant or chimera thereof, with or without a MiRP1 beta subunit or a mutant thereof, at a level effective to induce a pacemaker activity in the cell when implanted into a subject.

The present invention further provides a bypass bridge comprising gap junction-coupled stem cells of the invention, the bridge having a first end and a second end, both ends capable of being attached to two selected sites in a heart, so as to allow the propagation of an electrical signal across the tract between the two sites in the heart. In a specific embodiment of the invention, the first end is capable of being attached to the atrium and the second end capable of being attached to the ventricle, so as to allow propagation of a pacemaker and/or electrical current/signal from the atrium to travel across the tract to excite the ventricle.

In yet another embodiment of the invention, the stem cells of the bypass tract functionally express at least one protein selected from the group consisting of: a cardiac connexin; an alpha subunit and accessory subunits of a L-type calcium channel; an alpha subunit with or without the accessory subunits of a sodium channel; and a L-type calcium and/or sodium channel in combination with the alpha subunit of a potassium channel, with or without the accessory subunits of the potassium channel.

In another embodiment of the invention, the stem cells of the bypass bridge functionally expresses: (i) a hyperpolarization-activated, cyclic nucleotide-gated (HCN) ion channel capable of generating a pacemaker current in said cell, (ii) a chimeric HCN channel comprising an amino terminal portion, an intramembrane portion, and a carboxy terminal portion, wherein the portions are derived from more than one HCN isoform, and wherein the expressed chimeric HCN channel generates a pacemaker current in said cell, or (c) a mutant HCN channel wherein the mutant HCN channel generates a pacemaker current in said cell.

Further, the present invention provides the use of the stem cells of the invention in a tandem pacemaker system comprising (1) an electronic pacemaker; (2) a biological pacemaker comprising an implantable stem cell of the invention that functionally expresses (a) an HCN ion channel, or (b) a chimeric HCN channel, or (c) a mutant HCN channel wherein the expressed HCN, chimeric HCN or mutant HCN channel generates an effective pacemaker current when said cell is implanted into a subject's heart; (3) and/or a bypass bridge comprising a strip of gap junction-coupled stem cells having a first end and a second end, both ends capable of being attached to two selected sites in a heart, so as to allow the transmission of a pacemaker and/or electrical signal/current across the tract between the two sites in the heart.

The present invention provides methods for promoting cardiac repair in a subject, comprising administering to said subject an effective amount of the stem cells of the invention thereby promoting cardiac repair. The methods of the invention may be used to treat a variety of different cardiac disorders, including but not limited to, myocardial dysfunction or infarction, cardiac rhythm disorders, disorders at the sinoatrial node and disorders of the atrioventricular node. In patients in whom biological pacemaker activity and/or a bypass bridge has/have been provided, the subject may also be provided with an electronic pacemaker.

### EXAMPLE

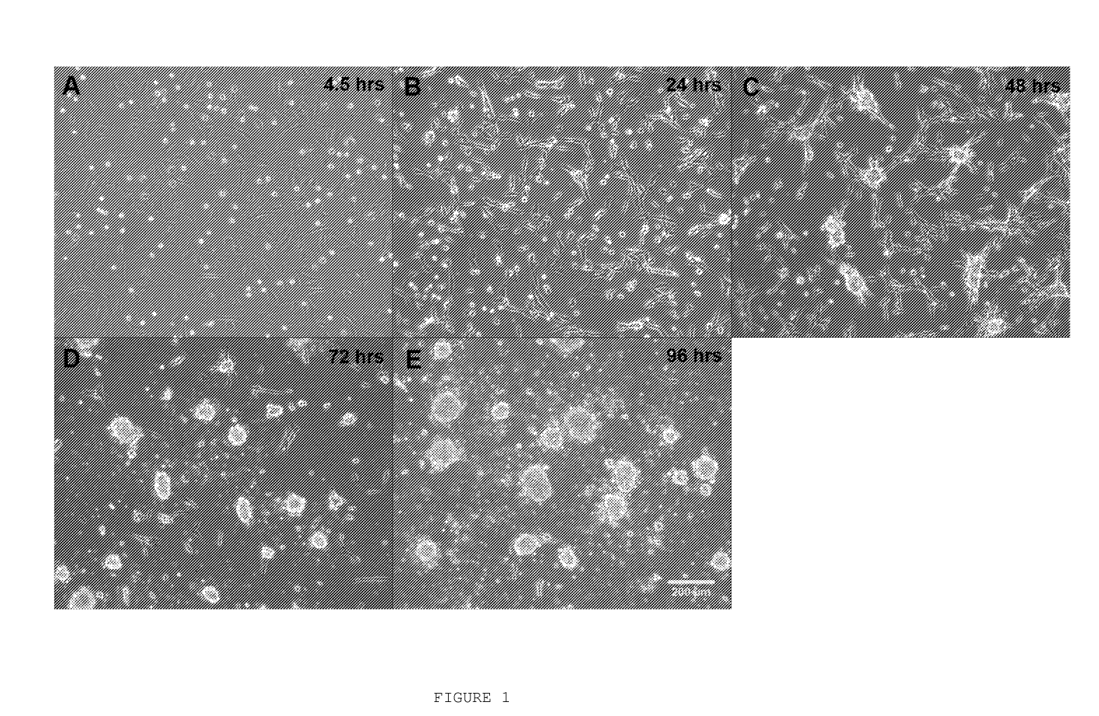

Representative images showing the formation of spheres from fibroblasts over time. FIG. 1A shows that the cells begin as an evenly distributed monolayer. B) By 24 hours after plating, the cells have undergone a morphological change. C) At 48 hours, the cells have begun to migrate toward one another, forming clusters. D) By 72 hours, the clusters pull together into spheres. Relatively few fibroblasts remain in the monolayer, indicating that most have entered spheres. E) After 4 days, the spheres are well-defined and growing in size.

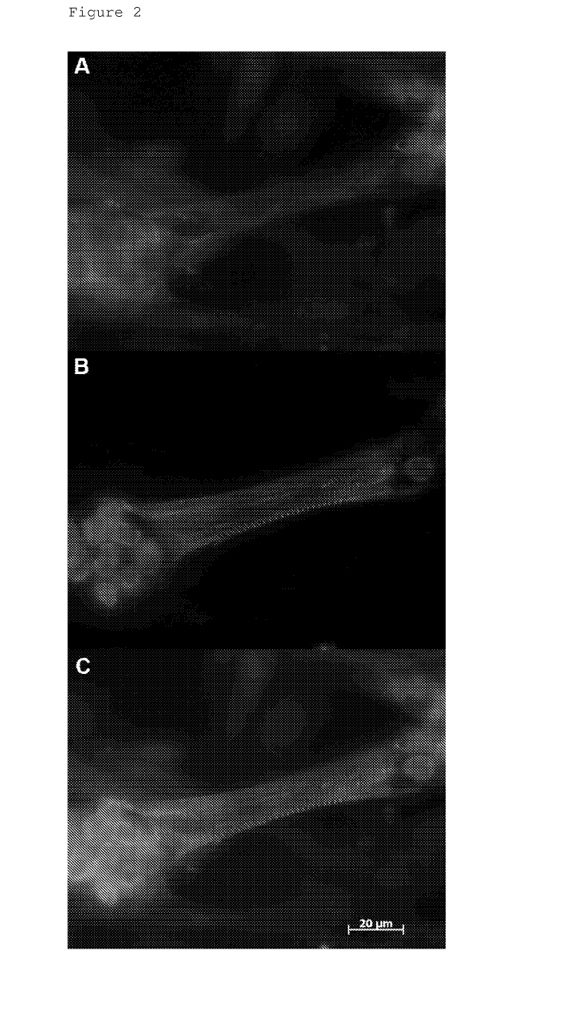

Cardiac differentiation of canine cardiosphere-derived cells (CFDCs). Cardiac explants were cultured for several days, during which time fibroblasts egressed from the explants and formed a monolayer on the bottom of the culture plate. The fibroblasts were collected and replated on a poly-D-lysine coated plate. After 2-3 days, the cells pulled together into clusters and lifted off of the plate as floating spheres (fibrospheres). These cardiac fibrospheres (CFDCs) were collected, dissociated to single cells, and labeled with DiI (orange). The labeled CFDCs were then placed in co-culture with neonatal rat ventricular myocytes (NRVMs) on segregated areas of a coverglass and allowed to share media for five days. The cells were then fixed and stained for the cardiac marker sarcomeric α-actinin (green). Nuclei were stained with DAPI (blue). FIG. 2A depicts DiI+cells within the CFDC area of the coverglass. FIG. 2B demonstrates sarcomeric α-actinin staining of the same cell showing distinct sarcomeric structure. FIG. 2C is a merged image showing co-localization of the DiI and sarcomeric α-actinin staining in a mono-nucleated CFDC. The presence of one nucleus indicates that cell fusion is not responsible for co-localization of the DiI and sarcomeric α-actinin.

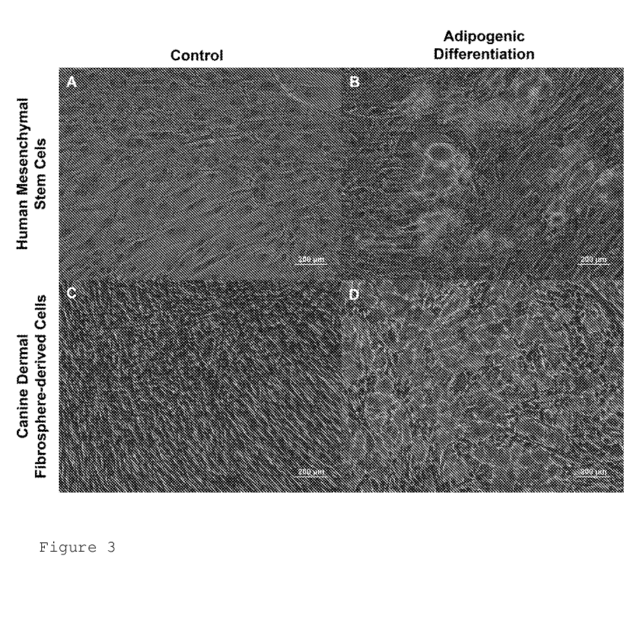

Adipogenic differentiation of canine dermal fibrosphere-derived cells (DFDCs). Canine dermal explants were kept in culture for several days until a monolayer of fibroblasts covered the bottom of the culture plate. The fibroblasts were collected and replated in poly-D-lysine coated plates, where they formed floating clusters of cells (fibrospheres) after 2-3 days. The fibrospheres were collected, dissociated and plated at confluence for adipogenic differentiation using a commercially available kit (Lonza, Walkersville, Md.). Adipogenic differentiation of DFDCs (FIG. 3D) and human mesenchymal stem cells (MSCs; FIG. 3B) is indicated by the appearance of fat vacuoles which stain red with Oil Red-0 stain. Human MSC and canine DFDC control cells (FIGS. 3A and 3C, respectively) that were not exposed to adipogenic media are also shown after Oil Red-0 staining.

What is claimed:

1. A method for the production of stem cells, of fibroblast origin, comprising:
(a) generating a culture of fibroblasts from a tissue sample; and
(b) culturing the fibroblasts in cell culture medium for a time sufficient to allow the dedifferentiation of said fibroblasts into stem cells, wherein said cell medium promotes the growth of the fibroblast in suspension.

2. The method of claim 1, further comprising the step of obtaining the stem cells by separating the stem cells from the culture medium.

3. The method of claim 1 wherein the tissue sample is derived from tissue of a patient to be treated with stem cells.

4. The method of claim 1 wherein the media is supplemented with one or more of the following growth factors: thrombin, basic fibroblast growth factor, epithelial growth factor, and cardiotrophin-1.

5. The method of claim 1 wherein the cells are grown in a culture vessel coated with poly-D-lysine.

6. The method of claim 1, further comprising the step wherein the stem cells are induced to differentiate along a desired lineage.

7. The method of claim 6, wherein the stem cells are induced to differentiate into cardiomyocytes, adipocytes, neurons, glia cells, endothelial cells, keratinocytes, hepatocytes or islet cells.

8. A stem cell derived from a dedifferentiated fibroblast.

9. The stem cell of claim 8, which has been induced to differentiate along a desired lineage.

10. The stem cell of claim 9, wherein the stem cell has been induced to differentiate into a cardiomyocyte, adipocyte, neuron, glia cell, endothelial cell, keratinocyte, hepatocyte or islet cell.

11. The stem cell of claim 8, genetically engineered to express a protein that provides a therapeutic benefit.

12. The stem cell of claim 11, genetically engineered to express a hyperpolarization-activated, cyclic nucleotide-gated (HCN) channel.

13. The stem cell of claim 12, further engineered to express a MiRP1 beta subunit.

14. The stem cell of claim 8 or 9, incorporated within a scaffold.

15. A pharmaceutical composition comprising the stem cell of claim 8 or 9 and a pharmaceutically acceptable carrier.

16. A method for promoting cardiac repair in a subject, comprising administration to said subject an effective amount of the stem cell of claim 8 or 9 into the heart.

17. The method of claim 16 wherein the subject has a disorder selected from the group consisting of myocardial dysfunction, myocardial infarction, cardiac rhythm disorder, a disorder of the sinoatrial node or atrioventricular node.

18. The method of claim 16 wherein the cells stimulate native cardiomyocyte proliferation.

19. A method for providing a biological pacemaker to a subject comprising administration of the cells of claim 12 or 13 into the heart.

20. A method for providing a bypass bridge comprising administration of a bridge having a first end and a second end, both ends capable of being attached to two selected sites in a heart, so as to allow the propagation of an electrical signal across the tract between the two sites in the heart, wherein said bridge comprises the stem cells of claim 8 or 9.

21. The method of claim 20 wherein the first end is capable of being attached to the atrium and the second end is capable of being attached to the ventricle.
